(19) United States
(12) Patent Application Publication
(10) Pub. No.: US 2009/0105338 A1
(43) Pub. Date: Apr. 23, 2009

(54) DEUTERIUM-ENRICHED GABEXATE MESYLATE

(75) Inventor: Anthony W. Czarnik, Reno, NV (US)

Correspondence Address: VANCE INTELLECTUAL PROPERTY, PC, 5467 HILL TOP STREET, CROZET, VA, 22932-3167, US

(73) Assignee: PROTIA, LLC, Reno, NV (US)

(21) Appl. No.: 11/874,330

(22) Filed: Oct. 18, 2007

(51) Int. Cl. A61K 31/225; A61P 1/18; C07C 303/26

(52) U.S. Cl. 514/548; 558/48

(57) ABSTRACT

The present application describes deuterium-enriched gabexate mesylate, pharmaceutically acceptable salt forms thereof, and methods of treating using the same.

### FIELD OF THE INVENTION

This invention relates generally to deuterium-enriched gabexate mesylate, pharmaceutical compositions containing the same, and methods of using the same.

### BACKGROUND OF THE INVENTION

Gabexate mesylate, shown below, is a well known synthetic protease inhibitor.

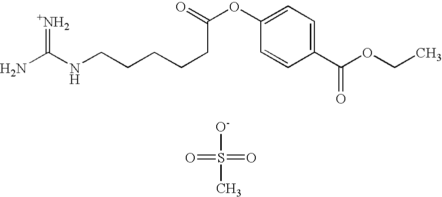

Since gabexate mesylate is a known and useful pharmaceutical, it is desirable to discover novel derivatives thereof.

### SUMMARY OF THE INVENTION

Accordingly, one object of the present invention is to provide deuterium-enriched gabexate mesylate.

It is another object of the present invention to provide pharmaceutical compositions comprising a pharmaceutically acceptable carrier and a therapeutically effective amount of at least one of the deuterium-enriched compounds of the present invention.

It is another object of the present invention to provide a method for treating pancreatitis comprising administering to a host in need of such treatment a therapeutically effective amount of at least one of the deuterium-enriched compounds of the present invention.

It is another object of the present invention to provide a novel deuterium-enriched gabexate mesylate for use in therapy.

It is another object of the present invention to provide the use of a novel deuterium-enriched gabexate mesylate for the manufacture of a medicament for the treatment of pancreatitis.

These and other objects, which will become apparent during the following detailed description, have been achieved by the inventor's discovery of the presently claimed deuterium-enriched gabexate mesylates.

### DETAILED DESCRIPTION OF PREFERRED EMBODIMENTS

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their non-enriched counterparts.

All percentages given for the amount of deuterium present are mole percentages.

It can be quite difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen will still be present. Deuterium-enriched can be achieved by either exchanging protons with deuterium or by synthesizing the molecule with enriched starting materials.

The present invention provides deuterium-enriched gabexate mesylate. There are twenty-four hydrogen atoms in the gabexate portion of gabexate mesylate as show by variables R₁—R₂₄ in formula I, below.

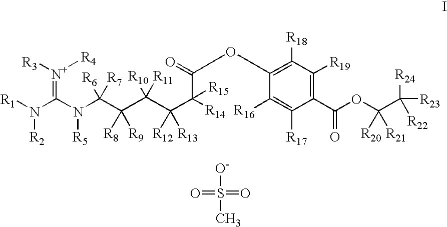

The hydrogens present on gabexate mesylate have different capacities for exchange with deuterium. The hydrogens represented by R₁—R₅ are the easiest to exchange with deuterium. The hydrogens represented by R₁₄—R₁₅ are exchangeable with deuterium in the presence of a strong acid or strong base. The hydrogens represented by R₁₆—R₁₉ are exchangeable with deuterium in the presence of a strong acid. The hydrogens represented by R₆—R₁₃ and R₂₀—R₂₄ are non-exchangeable. Deuterium enrichment at these positions must occur via a synthetic process (e.g., transesterification).

The present invention is based on increasing the amount of deuterium present in gabexate mesylate above its natural abundance. This increasing is called enrichment or deuterium-enrichment. If not specifically noted, the percentage of enrichment refers to the percentage of deuterium present in the composition. The amount of preferred enrichment is from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Since there are 24 hydrogens in the gabexate portion of gabexate mesylate, replacement of a single hydrogen atom on gabexate mesylate with deuterium would result in a molecule with about 4.2% deuterium enrichment. In order to achieve enrichment less than about 4.2%, but above the natural abundance, only partial deuteration of one site is required. Thus, less than about 4.2% enrichment would still refer to deuterium-enriched gabexate mesylate.

With the natural abundance of deuterium being 0.015%, one would expect that for approximately every 6,667 molecules of gabexate (1/0.00015=6,667), there is one naturally occurring molecule with one deuterium present. Since gabexate has 24 positions, one would roughly expect that for approximately every 160,000 molecules of gabexate (24×6,667), all 24 different, naturally occurring, mono-deuterated gabexates would be present. This approximation is a rough estimate as it doesn't take into account the different exchange rates of the hydrogen atoms on gabexate. For naturally occurring molecules with more than one deuterium, the numbers become vastly larger. In view of this natural abundance, the present invention, in an embodiment, relates to an amount of an deuterium enriched compound, whereby the enrichment recited will require more than naturally occurring deuterated molecules.

Examples of amounts include, but are not limited to (a) at least 0.01, 0.02, 0.03, 0.04, 0.05, 0.1, 0.2, 0.3, 0.4, 0.5, to 1 mole, (b) at least 0.1 moles, and (c) at least 1 mole of the compound. The present amounts also cover lab-scale (e.g., gram scale), kilo-lab scale (e.g., kilogram scale), and industrial scale (e.g., multi-kilogram or above scale) quantities as these will be more useful in the actual manufacture of a pharmaceutical. Industrial-scale refers to the amount of product that would be produced in a batch that was designed for clinical testing or sale/distribution to the public.

In view of the natural abundance of deuterium-enriched gabexate, the present invention also relates to isolated or purified deuterium-enriched gabextae. The isolated or purified deuterium-enriched gabexate is a group of molecules whose deuterium levels are above the naturally occurring levels (e.g., 4%). The isolated or purified deuterium-enriched gabextae can be obtained by techniques known to those of skill in the art (e.g., see the synthses described below).

The present invention also relates to compositions comprising deuterium-enriched gabextae. The compositions require the presence of deuterium-enriched gabexate which is greater than its natural abundance.

In another embodiment, the present invention provides a novel deuterium-enriched compound of formula I or a pharmaceutically acceptable salt thereof:

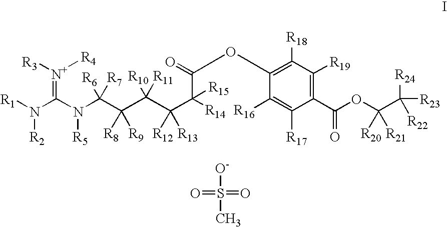

wherein R₁—R₂₄ are independently selected from H and D; and the abundance of deuterium in R₁—R₂₄ is at least 4%. The abundance can also be (a) at least 8%, (b) at least 16%, (c) at least 33%, (d) at least 50%, and (e) about 100%.

In another embodiment, the present invention provides a novel deuterium-enriched compound of formula I wherein the abundance of deuterium in R₆—R₁₅ is at least 10%. The abundance can also be (a) at least 20%, (b) at least 40%, (c) at least 60%, (d) at least 80%, and (e) about 100%.

In another embodiment, the present invention provides a novel deuterium-enriched compound of formula I wherein the abundance of deuterium in R₁—R₅ is at least 20%. The abundance can also be (a) at least 40%, (b) at least 60%, (c) at least 80%, and (d) about 100%.

In another embodiment, the present invention provides a novel deuterium-enriched compound of formula I wherein the abundance of deuterium in R₁—R₅ and R₁₆—R₁₉ is at least 67%. The abundance can also be (a) at least 78% and (b) about 100%.

In another embodiment, the present invention provides a novel deuterium-enriched compound of formula I wherein the abundance of deuterium in R₁—R₅ and R₁₄—R₁₉ is at least 55%. The abundance can also be (a) at least 64%, (b) at least 73%, (c) at least 82%, (d) at least 91%, and (e) about 100%.

In another embodiment, the present invention provides a novel deuterium-enriched compound of formula I wherein the abundance of deuterium in R₂₀—R₂₄ is at least 20%. The abundance can also be (a) at least 40%, (b) at least 60%, (c) at least 80%, and (d) about 100%.

In another embodiment, the present invention provides an isolated novel, deuterium enriched compound of formula I.

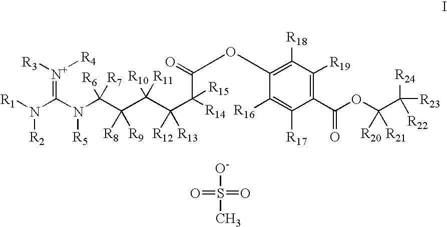

wherein R₁—R₂₄ are independently selected from H and D; and the abundance of deuterium in R₁—R₂₄ is at least 4%. The abundance can also be (a) at least 8%, (b) at least 16%, (c) at least 33%, (d) at least 50%, and (e) about 100%.

In another embodiment, the present invention provides an isolated novel, deuterium enriched compound of formula I, wherein the abundance of deuterium in R₆—R₁₅ is at least 10%. The abundance can also be (a) at least 20%, (b) at least 40%, (c) at least 60%, (d) at least 80%, and (e) about 100%.

In another embodiment, the present invention provides an isolated novel, deuterium enriched compound of formula I, wherein the abundance of deuterium in R₁—R₅ is at least 20%. The abundance can also be (a) at least 40%, (b) at least 60%, (c) at least 80%, and (d) about 100%.

In another embodiment, the present invention provides an isolated novel, deuterium enriched compound of formula I, wherein the abundance of deuterium in R₁—R₅ and R₁₆—R₁₉ is at least 67%. The abundance can also be (a) at least 78% and (b) about 100%.

In another embodiment, the present invention provides an isolated novel, deuterium enriched compound of formula I, wherein the abundance of deuterium in R₁—R₅ and R₁₄—R₁₉ is at least 55%. The abundance can also be (a) at least 64%, (b) at least 73%, (c) at least 82%, (d) at least 91%, and (e) about 100%.

In another embodiment, the present invention provides an isolated novel, deuterium enriched compound of formula I, wherein the abundance of deuterium in R₂₀—R₂₄ is at least 20%. The abundance can also be (a) at least 40%, (b) at least 60%, (c) at least 80%, and (d) about 100%.

In another embodiment, the present invention provides a novel mixture of deuterium-enriched compounds of formula I or a pharmaceutically acceptable salt thereof:

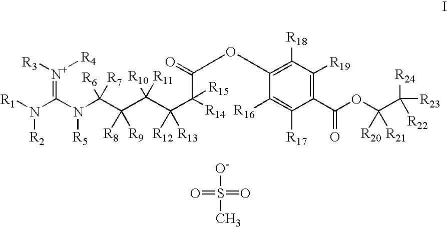

wherein R₁—R₂₄ are independently selected from H and D; and the abundance of deuterium in R₁—R₂₄ is at least 4%. The abundance can also be (a) at least 8%, (b) at least 16%, (c) at least 33%, (d) at least 50%, and (e) about 100%.

In another embodiment, the present invention provides a novel mixture of deuterium-enriched compounds of formula I wherein the abundance of deuterium in R₆—R₁₅ is at least 10%. The abundance can also be (a) at least 20%, (b) at least 40%, (c) at least 60%, (d) at least 80%, and (e) about 100%.

In another embodiment, the present invention provides a novel mixture of deuterium-enriched compounds of formula I wherein the abundance of deuterium in R₁—R₅ is at least 20%. The abundance can also be (a) at least 40%, (b) at least 60%, (c) at least 80%, and (d) about 100%.

In another embodiment, the present invention provides a novel mixture of deuterium-enriched compounds of formula I wherein the abundance of deuterium in R₁—R₅ and R₁₆—R₁₉ is at least 67%. The abundance can also be (a) at least 78% and (b) about 100%.

In another embodiment, the present invention provides a novel mixture of deuterium-enriched compounds of formula I wherein the abundance of deuterium in R₁—R₅ and R₁₄—R₁₉ is at least 55%. The abundance can also be (a) at least 64%, (b) at least 73%, (c) at least 82%, (d) at least 91%, and (e) about 100%.

In another embodiment, the present invention provides a novel mixture of deuterium-enriched compounds of formula I wherein the abundance of deuterium in R₂₀—R₂₄ is at least 20%. The abundance can also be (a) at least 40%, (b) at least 60%, (c) at least 80%, and (d) about 100%.

In another embodiment, the present invention provides novel pharmaceutical compositions, comprising: a pharmaceutically acceptable carrier and a therapeutically effective amount of a deuterium-enriched compound of the present invention.

In another embodiment, the present invention provides a novel method for treating pancreatitis, comprising: administering to a patient in need thereof a therapeutically effective amount of a deuterium-enriched compound of the present invention.

In another embodiment, the present invention provides an amount of a deuterium-enriched compound of the present invention as described above for use in therapy.

In another embodiment, the present invention provides an isolated deuterium-enriched compound of the present invention as described above for use in therapy.

In another embodiment, the present invention provides the use of an amount of a deuterium-enriched compound of the present invention for the manufacture of a medicament for the treatment of pancreatitis.

In another embodiment, the present invention provides the use of an isolated deuterium-enriched compound of the present invention for the manufacture of a medicament for the treatment of pancreatitis.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

### DEFINITIONS

The examples provided in the definitions present in this application are non-inclusive unless otherwise stated. They include but are not limited to the recited examples.

The compounds of the present invention may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. All processes used to prepare compounds of the present invention and intermediates made therein are considered to be part of the present invention. All tautomers of shown or described compounds are also considered to be part of the present invention.

“Host” preferably refers to a human. It also includes other mammals including the equine, porcine, bovine, feline, and canine families.

“Treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antiviral effect, or some other beneficial effect of the combination compared with the individual components.

### Synthesis

The compounds of the present invention can be prepared in a number of ways known to one skilled in the art of organic synthesis. The compounds of the present invention can be synthesized using the methods described below, together with synthetic methods known in the art of synthetic organic chemistry, or by variations thereon as appreciated by those skilled in the art. Preferred methods include those described below. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Wiley and Sons, 1991). All references cited herein are hereby incorporated in their entirety herein by reference.

Gabexate mesylate can be prepared according to well known procedures (see, for example, JP-62059254). Below is an example of how to prepare gebexate mesylate.

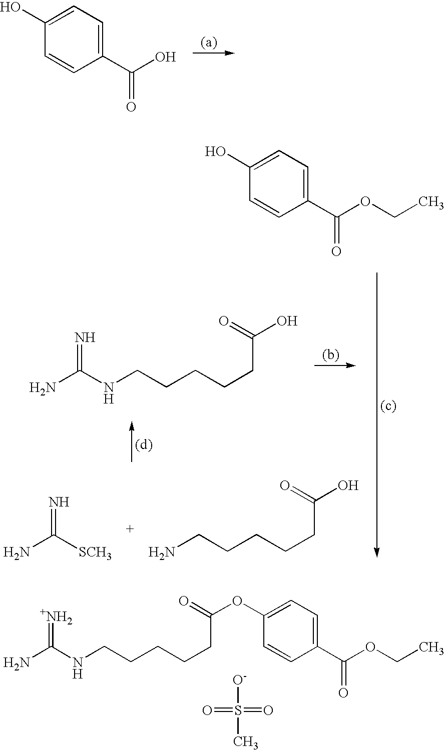

In step (a), 4-hydroxy-benzoic acid can be converted to ethyl 4-hydroxy-benzoate by esterifying with ethanol in the presence of an acid catalyst (e.g., H₂SO₄). It can be beneficial to heat the reaction up to the boiling point of the solvent (e.g., ethanol). In step (b), 6-guanidino-hexanoic acid can be treated with an acid activator (e.g., ClCO₂Et) and a base (e.g., Et₃N), preferably in an aprotic solvent (e.g., dimethylformamide) and also preferably in an ice-water bath. In step (c), after the caproic acid has been activated, it can then be treated with ethyl 4-hydroxy-benzoate, preferably in the presence of additional base (e.g., Et₃N). The final product can then be obtained by treating the resulting ester with MeSO₃H. Step (d) shows how 6-guanidinohexanoic acid could be formed from 2-methyl-isothiourea and 6-amino-hexanoic acid. It is also noted that 6-amino-hexanoic acid can be formed from 6-carbamoyl-hexanoic acid via a degradation reaction (e.g., Hofmann degradation with Br₂ and NaOH).

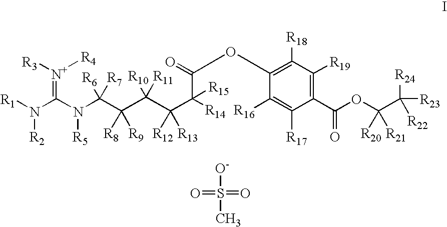

The desired site of deuterium-enrichment will influence the synthesis of the compound. As noted previously, gabexate mesylate (formula I above) has 24 hydrogens. The hydrogens present on gabexate mesylate have different capacities for exchange with deuterium. The hydrogens represented by R₁—R₅ are the easiest to exchange with deuterium. These positions could be enriched simply by stirring gabexate mesylate in the presence of D₂O. They are also the most readily exchanged under physiological conditions. Thus, when any of R₁—R₅ are deuterium atoms, it is expected that they will readily exchange with protons after administration to a patient.

The hydrogens represented by R₁₄—R₁₅ are exchangeable with deuterium in the presence of a strong acid (e.g., D₂SO₄/D₂O and room temperature) or strong base (e.g., Li—O-t-Bu/DO-t-Bu). Treatment with a strong acid should also cause the exchange of R₁—R₅ and potentially R₁₆—R₁₉. Since gabexate contains an acid-sensitive guanidine group as well as two hydrolysable esters, it is probable that treatment with a strong acid or strong base would hydrolyze or decompose the gabexate molecule. Therefore, it is preferred that the R₁₄—R₁₅ sites be enriched prior to synthesis of the gabexate molecule. This can be accomplished by enriching the α-hydrogens of the hexanoic acid prior to addition of the guanidine moiety. For example, hexanoic acid with an appropriate group at the 6 position can be treated with either a strong acid (e.g., D₂SO₄/D₂O) or strong base (e.g., Li—O-t-Bu/DO-t-Bu). The resulting enriched molecule can then be modified to achieve the 6-guanidino-2,2-dideutero-hexanoic acid starting material.

The hydrogens represented by R₁₆—R₁₉ are exchangeable with deuterium in the presence of a strong acid (e.g., D₂SO₄/D₂O). Due to the sensitivity of the gabexate molecule, it is preferred that the R₁₆—R₁₉ hydrogens be exchanged in the starting material. For example, 2,3,5,6-tetradeutero-4-hydroxybenzoic acid or the fully deuterated version can be used as a starting material in step (a) of the above described synthesis. Both of these starting materials could be obtained by treating of 4-hydroxybenzoic acid with a strong acid (e.g., D₂SO₄/D₂O).

The hydrogens represented by R₆—R₁₃ are non-exchangeable. Deuterium enrichment of R₆—R₁₃ can only occur by using deuterated starting materials. (e.g., deuterated 6-guanidinohexanoic acid). An appropriately 6-substituted-deuterated hexanoic acid could be used as the starting material to make the 6-guanidinohexanoic acid used in the above-described synthesis.

The hydrogens represented by R₂₀—R₂₄ are non-exchangeable. Deuterium enrichment of R₂₀—R₂₄ can be achieved by esterification/transesterification with a deuterium-enriched ethanol or deuterium-enriched ethoxide (e.g., deuterium-enriched sodium or potassium ethoxide). The enrichment of R₂₀—R₂₄ can be done at any step of the synthesis. For example, in step (a) of the above synthesis, the esterification of 4-hydroxbenzoic acid could be performed with deuterated ethanol (e.g., pentadeutero or hexadeutero-ethanol). If however it is desired, then a transesterification can be peformed at a later stage. It may be desireable to use a deuterated acid in the esterification/transesterification (e.g., D₂SO₄). Use of a deuterated acid may cause exchange of the other exchangable protons.

Examples of deuterated starting materials using for making compounds of the present invention and suggested routes for making these materials are shown below.

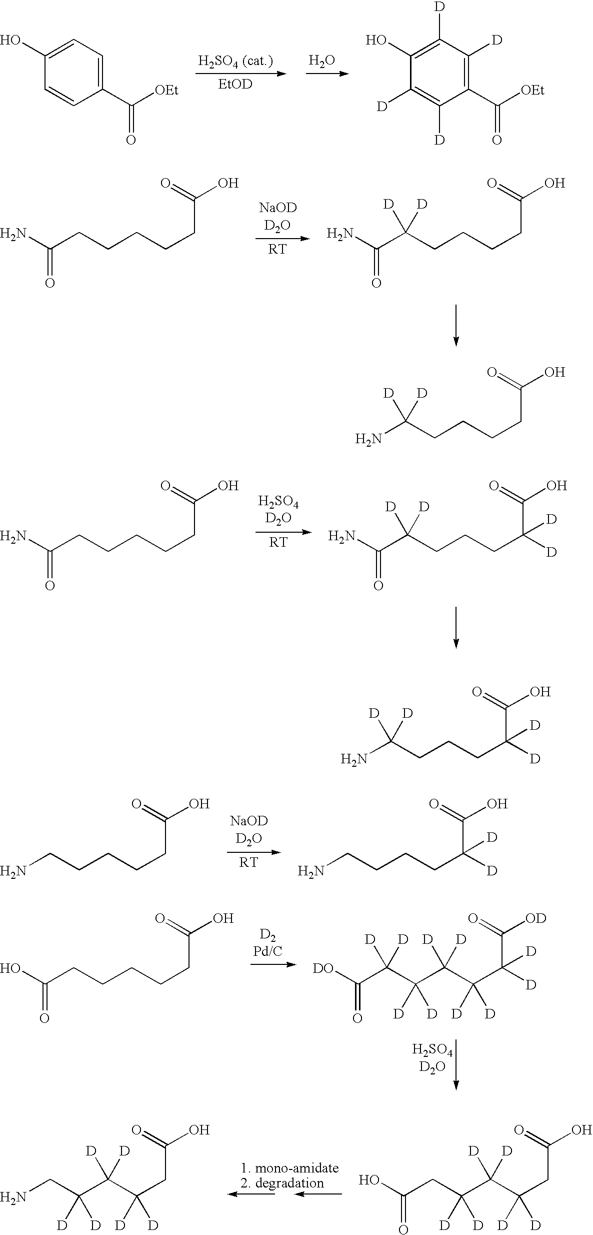

Other features of the invention will become apparent in the course of the following descriptions of exemplary embodiments that are given for illustration of the invention and are not intended to be limiting thereof.

### EXAMPLES

Table 1 provides compounds that are representative examples of the present invention.
When one of R₁-R₂₄ is present, it is selected from H or D.

| | |
|---|---|
| 1 | 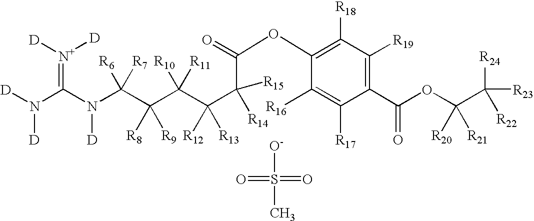 |
| 2 | 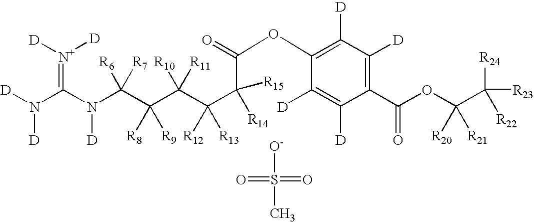 |
| 3 | 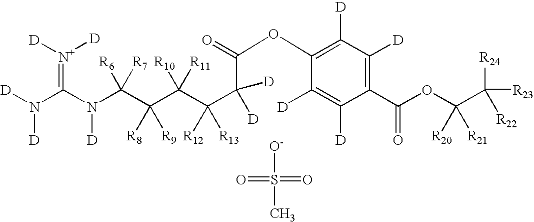 |
| 4 | 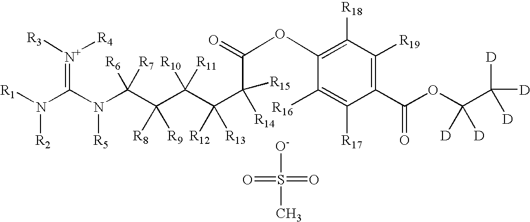 |
| 5 | 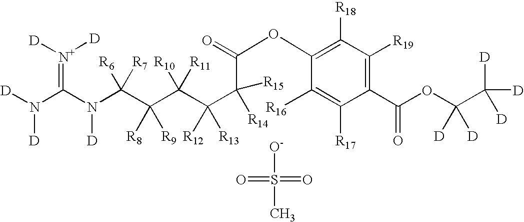 |
| 6 | 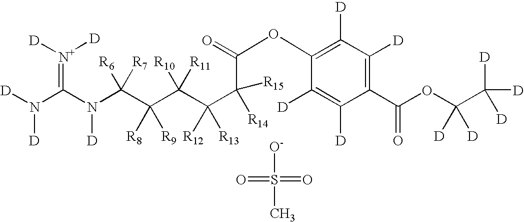 |
| 7 | 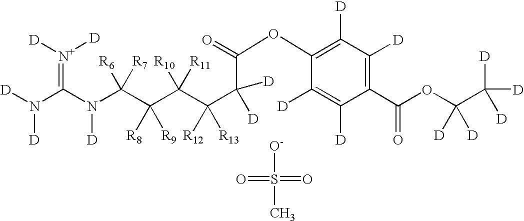 |
| 8 | 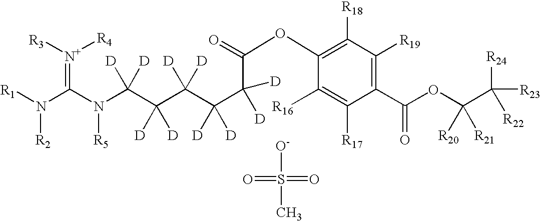 |
| 9 | 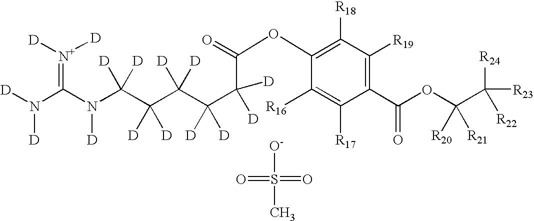 |
| 10 | 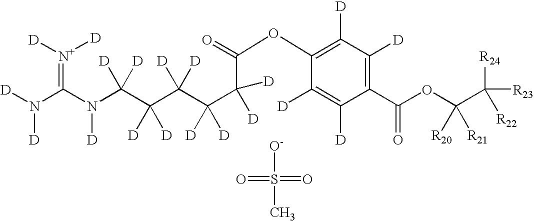 |
| 11 | 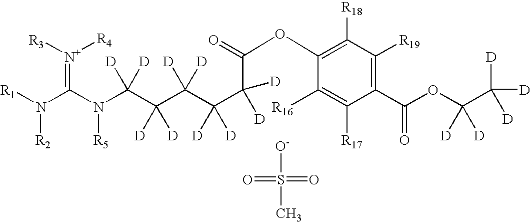 |
| 12 | 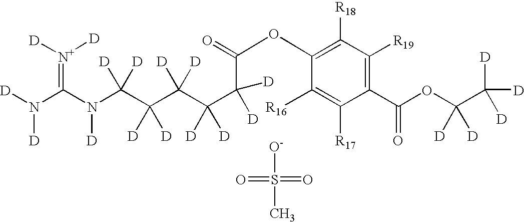 |
| 13 | 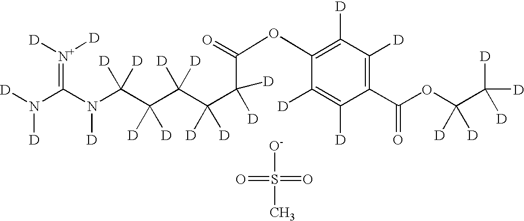 |

Table 2 provides compounds that are representative examples of the present invention.
Where H is shown, it represents naturally abundant hydrogen.

| | |
|---|---|
| 14 | 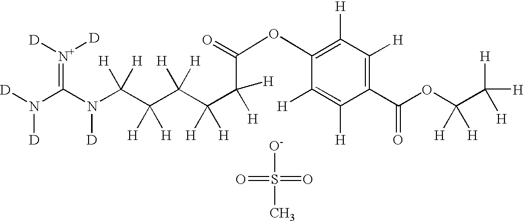 |
| 15 | 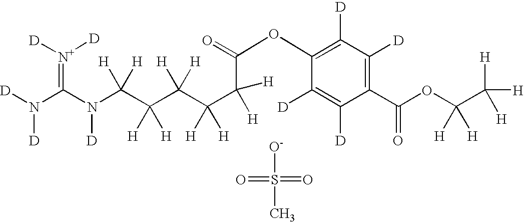 |
| 16 | 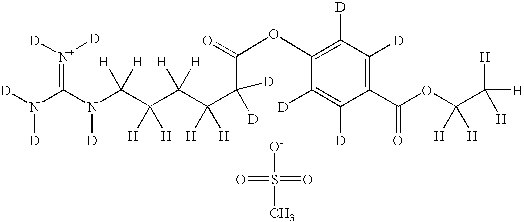 |
| 17 | 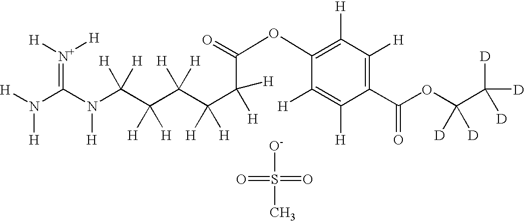 |
| 18 | 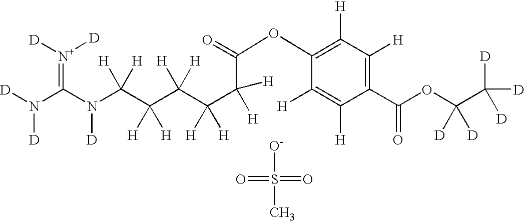 |
| 19 | 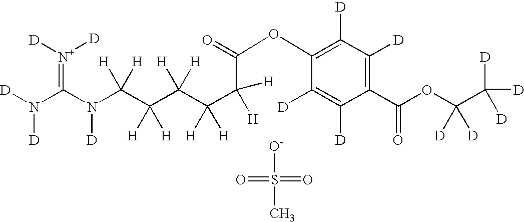 |
| 20 | 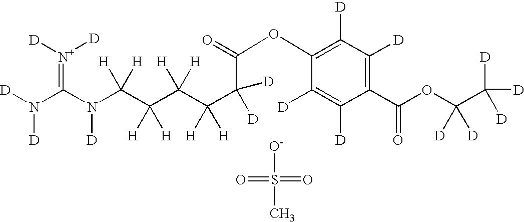 |
| 21 | 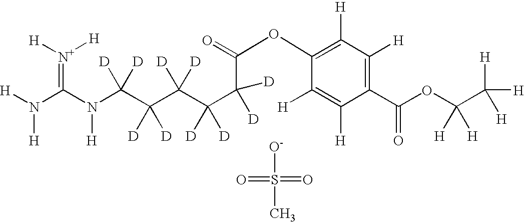 |
| 22 | 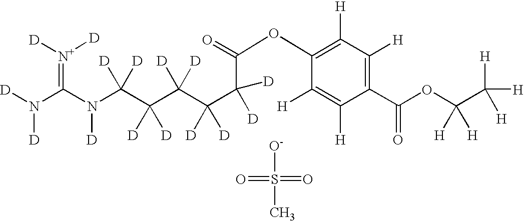 |
| 23 | 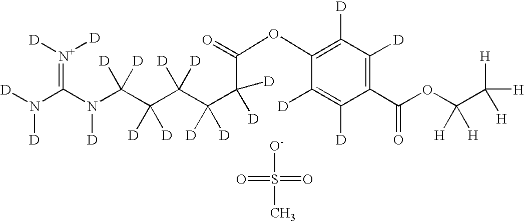 |
| 24 | 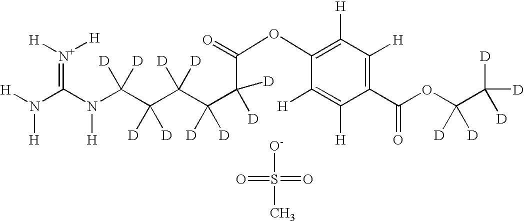 |
| 25 | 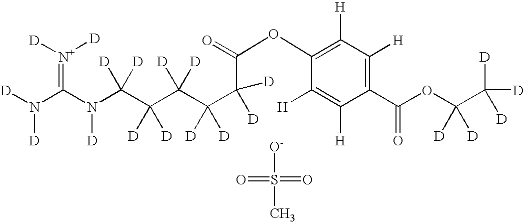 |
| 26 | 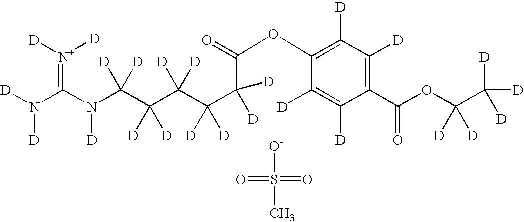 |

Numerous modifications and variations of the present invention are possible in light of the above teachings. It is therefore to be understood that within the scope of the appended claims, the invention may be practiced otherwise that as specifically described herein.

What is claimed is:

1. A deuterium-enriched compound of formula I or a pharmaceutically acceptable salt thereof: 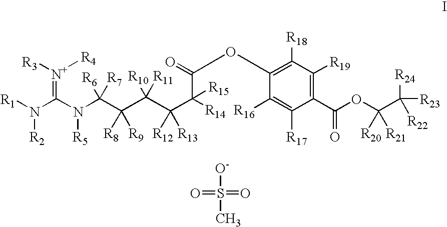
wherein R₁—R₂₄ are independently selected from H and D; and the abundance of deuterium in R₁—R₂₄ is at least 4%.

2. A deuterium-enriched compound of claim 1, wherein the abundance of deuterium in R₁—R₂₄ is selected from (a) at least 8%, (b) at least 16%, (c) at least 33%, (d) at least 50%, and (e) 100%.

3. A deuterium-enriched compound of claim 1, wherein the abundance of deuterium in R₆—R₁₅ is selected from (a) at least 10%, (b) at least 20%, (c) at least 40%, (d) at least 60%, (e) at least 80%, and (f) 100%.

4. A deuterium-enriched compound of claim 1, wherein the abundance of deuterium in R₁—R₅ is selected from (a) at least 20%, (b) at least 40%, (c) at least 60%, (d) at least 80%, and (e) 100%.

5. A deuterium-enriched compound of claim 1, wherein the abundance of deuterium in R₁—R₅ and R₁₆—R₁₉ is selected from (a) at least 67%, (b) at least 78%, and (c) 100%.

6. A deuterium-enriched compound of claim 1, wherein the abundance of deuterium in R₁—R₅ and R₁₄—R₁₉ is selected from (a) at least 55%, (b) at least 64%, (c) at least 82%, and (d) 100%.

7. A deuterium-enriched compound of claim 1, wherein the abundance of deuterium in R₂₀—R₂₄ is selected from (a) at least 50% and (b) 100%.

8. A deuterium-enriched compound of claim 1, wherein the compound is selected from compounds 1-13 of Table 1 and compounds 14-26 of Table 2:

9. An isolated deuterium-enriched compound of formula I or a pharmaceutically acceptable salt thereof: 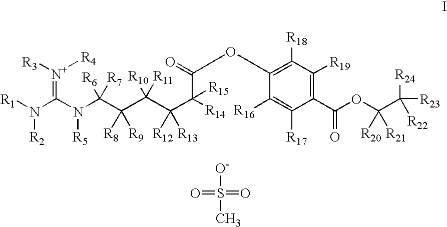
wherein R₁—R₂₄ are independently selected from H and D; and
the abundance of deuterium in R₁-R₂₄ is at least 4%.

10. An isolated deuterium-enriched compound of claim 9, wherein the abundance of deuterium in R₁—R₂₄ is selected from (a) at least 8%, (b) at least 16%, (c) at least 33%, (d) at least 50%, and (e) 100%.

11. An isolated deuterium-enriched compound of claim 9, wherein the abundance of deuterium in R₆—R₁₅ is selected from (a) at least 10%, (b) at least 20%, (c) at least 40%, (d) at least 60%, (e) at least 80%, and (f) 100%.

12. An isolated deuterium-enriched compound of claim 9, wherein the abundance of deuterium in R₁—R₅ is selected from (a) at least 20%, (b) at least 40%, (c) at least 60%, (d) at least 80%, and (e) 100%.

13. An isolated deuterium-enriched compound of claim 9, wherein the abundance of deuterium in R₁—R₅ and R₁₆—R₁₉ is selected from (a) at least 67%, (b) at least 78%, and (c) 100%.

14. An isolated deuterium-enriched compound of claim 9, wherein the abundance of deuterium in R₁—R₅ and R₁₄—R₁₉ is selected from (a) at least 55%, (b) at least 64%, (c) at least 82%, and (d) 100%.

15. An isolated deuterium-enriched compound of claim 9, wherein the abundance of deuterium in R₂₀—R₂₄ is selected from (a) at least 50% and (b) 100%.

16. An isolated deuterium-enriched compound of claim 9, wherein the compound is selected from compounds 1-13 of Table 1 and compounds 14-26 of Table 2:

17. A mixture of deuterium-enriched compounds of formula I or a pharmaceutically acceptable salt thereof: 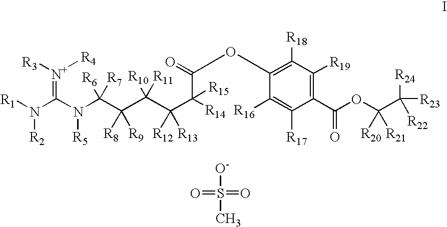
wherein R₁—R₂₄ are independently selected from H and D; and
the abundance of deuterium in R₁—R₂₄ is at least 4%.

18. A mixture of deuterium-enriched compounds of claim 17, wherein the abundance of deuterium in R₁—R₂₄ is selected from (a) at least 8%, (b) at least 16%, (c) at least 33%, (d) at least 50%, and (e) 100%.

19. A mixture of deuterium-enriched compounds of claim 17, wherein the abundance of deuterium in R₆—R₁₅ is selected from (a) at least 10%, (b) at least 20%, (c) at least 40%, (d) at least 60%, (e) at least 80%, and (f) 100%.

20. A mixture of deuterium-enriched compounds of claim 17, wherein the abundance of deuterium in R₁—R₅ is selected from (a) at least 20%, (b) at least 40%, (c) at least 60%, (d) at least 80%, and (e) 100%.

21. A mixture of deuterium-enriched compounds of claim 17, wherein the abundance of deuterium in R₁—R₅ and R₁₆—R₁₉ is selected from (a) at least 67%, (b) at least 78%, and (c) 100%.

22. A mixture of deuterium-enriched compounds of claim 17, wherein the abundance of deuterium in R₁—R₅ and R₁₄—R₁₉ is selected from (a) at least 55%, (b) at least 64%, (c) at least 82%, and (d) 100%.

23. A mixture of deuterium-enriched compounds of claim 17, wherein the compound is selected from compounds 1-13 of Table 1 and compounds 14-26 of Table 2:

24. A pharmaceutical composition, comprising: a pharmaceutically acceptable carrier and a therapeutically effective amount of a compound of claims 1-23 or a pharmaceutically acceptable salt form thereof.

25. A method for treating pancreatitis, comprising: administering, to a patient in need thereof, a therapeutically effective amount of a compound of claims 1-23 or a pharmaceutically acceptable salt form thereof.
